(19) United States
(12) Patent Application Publication
(10) Pub. No.: US 2023/0133133 A1
(43) Pub. Date: May 4, 2023

(54) ALDEHYDE GENERATION VIA ALKENE HYDROFORMYLATION

(71) Applicant: NORTH CAROLINA STATE UNIVERSITY, Releigh, NC (US)

(72) Inventors: Milad Abolhasani, Raleigh, NC (US); Keshav Raghuvanshi, Varanasi, Uttar Pradesh (IN); Cheng Zhu, Richland, WA (US); Mahdi Ramezani, Raleigh, NC (US); Erik E. Santiso, Raleigh, NC (US); Stefano Menegatti, Raleigh, NC (US)

(21) Appl. No.: 17/912,211

(22) PCT Filed: Mar. 19, 2021
(86) PCT No.: PCT/US21/23224

§ 371 Date: Sep. 16, 2022

Related U.S. Application Data
(60) Provisional application No. 62/991,783, filed on Mar. 19, 2020.

(51) Int. Cl. C07C 45/50; B01J 19/24; B01J 31/24

(52) U.S. Cl. CPC C07C 45/50; B01J 19/244; B01J 31/24; B01J 2219/00162; B01J 2219/00054; B01J 2219/00132

(57) ABSTRACT

Aldehyde generation includes providing a first input stream, a second input, and an alkene substrate to a reactor system. The first input stream includes a catalyst, a ligand, and an organic solvent. The second input stream includes a mixture of carbon monoxide (CO) and hydrogen gas (H₂). The alkene substrate is in either gaseous form or liquid form, the liquid form of the alkene substrate being provided with the first input stream, the gaseous form of the alkene substrate being provided with the second input stream. The reactor system includes a first reactor and a second reactor, where the second reactor is gas permeable and positioned within the first reactor.

### CROSS-REFERENCE TO RELATED APPLICATIONS

The present application is related to and claims the priority benefit of U.S. Provisional Patent Application No. 62/991,783, filed November Mar. 19, 2020, the entire contents of which are incorporated herein by reference.

### TECHNICAL FIELD

The present disclosure relates to systems and methods for alkene hydroformylation. More particularly, systems and methods disclosed and contemplated herein can be configured for continuous aldehyde generation.

### INTRODUCTION

Hydroformylation is an example of homogenous catalysis and can be used in aldehyde generation. Two types of hydroformylation include cobalt-catalyzed hydroformylation and rhodium (Rh)-catalyzed hydroformylation, known as low-pressure-oxo (LPO), which operates at syngas pressures ranging between 10-60 bar. Regioselectivity is a facet of hydroformylation that can reduce costs of separation or purification of product aldehydes. One example of a highly regioselective ligand is 2,2′-Bis(diphenylphosphinomethyl)-1,1′-bipheny (BISBI), a bidentate bisphosphine chelating ligand that can be used during production of linear aldehydes.

### SUMMARY

The instant disclosure is directed to aldehyde generation using alkene hydroformylation. In one aspect, a method for generating aldehydes includes providing a first input stream to a reactor system, providing a second input stream to the reactor system, and providing an alkene substrate to the reactor system. The method can also include monitoring a temperature within the first reactor, controlling a heating source such that the temperature within the reactor system is 80° C. to 120° C., controlling a pressure within the reactor system to be less than 150 psig, controlling a first input stream flow rate and a second input stream flow rate such that a residence time in the second reactor is 1 second to 3 hours, and generating a reactor output stream including the aldehydes.

The first input stream can include a catalyst, a ligand, and an organic solvent. The second input stream can include a mixture of carbon monoxide (CO) and hydrogen gas (H₂). The alkene substrate can be provided in either gaseous form or liquid form, the liquid form of the alkene substrate being provided with the first input stream, the gaseous form of the alkene substrate being provided with the second input stream. The reactor system includes a first reactor and a second reactor, where the second reactor is gas permeable and positioned within the first reactor. The first reactor is gas-impermeable.

There is no specific requirement that a system, technique or method relating to alkene hydroformylation include all of the details characterized herein, in order to obtain some benefit according to the present disclosure. Thus, the specific examples characterized herein are meant to be exemplary applications of the techniques described, and alternatives are possible.

### BRIEF DESCRIPTION OF THE DRAWINGS

FIG. 1 is a schematic diagram of an example aldehyde generation system.

FIG. 2 is a cut-away view showing a portion of a tube-in-tube reactor usable in the aldehyde generation system shown in FIG. 1.

FIG. 3 shows a perspective, partially exploded view of an example heating system usable in the aldehyde generation system shown in FIG. 1.

FIG. 4 shows a perspective view of an example heating system usable in the aldehyde generation system shown in FIG. 1.

FIG. 5 shows an example method for generating aldehydes.

FIG. 6 is a schematic diagram of an experimental system used to generate aldehydes.

FIG. 7 and FIG. 8 show nuclear magnetic resonance (NMR) spectroscopy images for an experimental hydroformylation of propylene.

FIG. 9 shows a possible mechanism of Rh-catalyzed hydroformylation of alkenes in a tube-in-tube reactor using BISBI as the ligand.

FIG. 10 shows hydrogen-deuterium (H/D) scrambling study of Rh-catalyzed hydroformylation of alkenes in a tube-in-tube flow reactor using deuterated styrene (7) and styrene (9) as substrates, and BISBI as ligand.

### DETAILED DESCRIPTION

Systems and methods disclosed and contemplated herein relate to aldehyde generation using alkene hydroformylation. In some instances, systems and methods disclosed herein can be configured for continuous synthesis of aldehydes, at low syngas pressures, using homogenous rhodium (Rh)-catalyzed reactions.

Exemplary reactor systems include tube-in-tube reactor configurations, where a gas-permeable tube is enclosed within a gas-impermeable tube. Generally, a first input stream and a second input stream are provided to exemplary reactor systems. Exemplary first input streams can include catalyst, ligand, and organic solvent. Exemplary second input streams can include carbon monoxide (CO), hydrogen gas (H₂). An alkene substrate is provided as a liquid alkene substrate in the first input stream or as gas alkene substrate in the second input stream, depending upon whether the alkene substrate is a liquid or a gas at ambient or near-ambient conditions. These and other aspects are discussed in the following sections.

### I. Definitions

Unless otherwise defined, all technical and scientific terms used herein have the same meaning as commonly understood by one of ordinary skill in the art. In case of conflict, the present document, including definitions, will control. Example methods and materials are described below, although methods and materials similar or equivalent to those described herein can be used in practice or testing of the present disclosure. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

### II. Exemplary Chemical Aspects

Example systems and methods involve input streams provided to reactor systems and output streams generated by reactor systems. The sections below discuss various chemical aspects of exemplary systems and methods.

### A. Example Input Streams

As mentioned above, exemplary reactor systems can receive first input streams and second input streams. Generally, exemplary first input streams are liquid phase and exemplary second input streams are gaseous phase.

Usually, alkene substrate is provided to exemplary reactor systems as either a liquid form or a gas. Alkene substrate is typically provided at ambient or near-ambient conditions (roughly, 1 atm and 20-25° C.). Alkenes that are liquid at ambient or near-ambient conditions can be provided in first input streams, which can be liquid. Alkenes that are gaseous at ambient or near ambient conditions can be provided in second input streams, which can be gaseous.

Example first input streams can include catalyst, ligand, and organic solvent. In some instances, exemplary first input streams can include liquid alkene substrate. In some instances, catalyst and ligand are provided from a first source and liquid alkene substrate and organic solvent are provided from a second source.

Various catalysts can be used in exemplary systems. For instance, the catalyst may be rhodium, iron, or cobalt.

Various ligands can be used in exemplary systems. Typically, ligands are selected that are regioselective or highly regioselective. Highly regioselective ligands are those that have linear to branch regioselectivity values no less than 10. In some instances, exemplary ligands are fluorophosphite ligands, phosphine ligands, or phosphite ligands. For instance, ligands can include 2,2′-Bis(diphenylphosphinomethyl)-1,1′-bipheny (BISBI), a bidentate bisphosphine chelating ligand.

Various alkene substrates can be used and can be selected based on desired aldehyde products generated by exemplary systems. Exemplary alkene substrates may be liquid or gas under ambient conditions. Typically, alkene substrates are C₃-C₈ alkenes. Generally, C₃ and C₄ alkenes are gaseous at ambient conditions and C₅-C₈ alkenes are liquid at ambient conditions. Exemplary alkene substrates can include 1-octene, hexene, pentene, and propylene.

Various organic solvents can be used in exemplary systems. For instance, organic solvents can include toluene, hexane, xylene, and benzene.

Example second input streams can include carbon monoxide (CO) and hydrogen gas (H₂). In some instances, second input streams can include gaseous alkene substrate. In some instances, carbon monoxide (CO) and hydrogen gas (H₂) are provided from different sources, where at least one, but in some instances both, sources have mass flow controllers to alter a ratio of CO/H₂ in the second input stream.

Various molar ratios of CO/H₂ are possible in exemplary second input streams. For example, molar ratios of CO/H₂ can range between 10:1-1:1-1:0 in the second input streams. As examples, molar ratios of CO/H₂ can be 10:1; 9:1; 8:1; 7:1; 6:1; 5:1; 4:1; 3:1; 2:1; 1.5:1; 1:1; 1:0.75; 1:0.5; 1:0.25; 1:0.1; or 1:0.

### B. Example Reactor Products

Exemplary systems and methods can generate various aldehydes, depending upon the selection of the catalyst, the ligand, and/or the alkene substrate. For instance, normal (n) and iso-(i) aldehydes can be generated. Examples can include, but are not limited to, C₄ aldehydes (n-butyraldehyde and i-butyraldehyde), and C9 aldehydes (nonal and 2-methyloctanal).

In various implementations, generated aldehydes have a linear to branch aldehyde ratio greater than 10; greater than 12; greater than 15; greater than 17; or greater than 20.

Various implementations can have different reactions, depending upon, for instance, the catalyst, the ligand, and the alkene substrate. Scheme I below shows an example reaction for Rh-catalyzed-hydrofomylation of 1-octene.

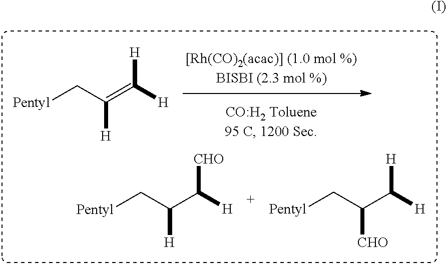

### III. Example Reactor Systems

Exemplary reactor systems disclosed and characterized herein can operate under temperatures and pressures sufficient for continuous aldehyde production. Typically, exemplary reactor systems operate under lower pressures than found in existing aldehyde generation systems.

### A. Example Reactor Configurations

Exemplary systems have a reactor-in-reactor configuration, where the reactors may have a tubular shape. In some implementations, exemplary reactor systems include a single tube-in-tube reactors. In some implementations, exemplary reactor systems include a plurality of tube-in-tube reactors.

Exemplary systems with multiple reactors typically have those reactors arranged in parallel. In some instances, exemplary systems with multiple reactors have identically-sized reactors.

Typically, the first reactor and the second reactor can be arranged for co-current flow. In some instances, the first reactor and the second reactor can be arranged for counter-current flow.

Exemplary reactor systems have a first reactor and a second reactor, where the second reactor is positioned within the first reactor. The first reactor is gas-impermeable. A commercially-available example material usable for the first reactor material can be stainless steel or fluoropolymer tubing such as fluorinated ethylene propylene (FEP) tubing (Altaflo, Sparta, N.J.), Perfluoroalkoxy (PFA), or Polytetrafluoroethylene (PTFE).

Exemplary first reactors can have various diameters. In exemplary implementations, first reactors may have an outer diameter of ⅛ inch and an inner diameter of 1/16 inch. Other diameters are contemplated.

The second reactor is gas-permeable and enables gas provided to the second reactor to permeate into the first reactor. The second reactor material can be made of highly gas-premable perfluoropolymer membranes. A commercially-available example material usable for gas-permeable reactors is Teflon AF 2400 (Chemours, Wilmington, Del.).

Exemplary second reactors can have various diameters. In exemplary implementations, second reactors may have an outer diameter of 0.04 inch and an inner diameter of 0.032 inch. Other diameters are contemplated.

Exemplary reactor systems can have various channel lengths. Typically, a channel length of the first reactor is the same as a channel length of the second reactor. Reactor lengths can be configured to, in combination with flow rates, achieve desired residence times. Example reactor lengths include, 1.75 m, 1.90 m, 2.0 m, 2.1 m, 2.2 m, 2.25 m, 2.3 m, 2.4 m, or 2.5 m. Other reactor lengths are contemplated.

Exemplary reactor systems include one or more heating systems to control heat within the reactors. In some implementations, exemplary heating systems include a first plate and a second plate, where the reactor system is disposed between the first plate and the second plate. In some instances, a plurality of first and second plates can be provided in a stacked arrangement, particularly for implementations with a plurality of reactor systems that may be arranged in parallel flow.

Exemplary heating systems can have a variety of configurations and various materials of construction. For instance, example first plate and second plates may include channels sized to hold reactor tubes. In some instances, example first and second plates may be a metal material, such as aluminum.

Exemplary heating systems can be heated in a variety of manners. For instance, one or more capillary heaters may be provided in the first plate. In some instances, or more capillary heaters may be provided in the first plate and in the second plate.

### B. Example Operating Characteristics

Operating pressures of exemplary reactor systems are typically no greater than 500 psig. For instance, an operating pressure of an exemplary reactor system can be less than 150 psig, less than 110 psig, or less than 50 psig. In various implementations, operating pressures of exemplary reactor systems are less than 500 psig; less than 400 psig; less than 350 psig; less than 300 psig; less than 250 psig; less than 200 psig; less than 150 psig; less than 110 psig; less than 100 psig; less than 75 psig; less than 70 psig; less than 65 psig; less than 60 psig; less than 55 psig; or less than 50 psig. In some instances, operating pressures of exemplary reactor systems are between 50 psig to 75 psig; between 50 psig and 100 psig; between 75 psig to 110 psig; between 100 psig to 150 psig; between 75 psig to 150 psig; between 150 psig and 250 psig; and between 250 psig and 400 psig.

Temperatures within the reactor system are typically less than 120° C. For instance, temperatures within the reactor system can be between 80° C. to 120° C. In various implementations, temperatures within the reactor system can be 80° C. to 120° C.; 80° C. to 110° C.; 90° C. to 120° C.; 80° C. to 100° C.; 90° C. to 110° C.; 100° C. to 120° C.; 85° C. to 95° C.; 90° C. to 105° C.; 105° C. to 120° C.; 90° C. to 100° C.; 100° C. to 110° C.; or 110° C. to 120° C.

Residence times in exemplary reactors can vary based on the flowrate of liquid stream and the reactor volume, and can be influenced by reactor temperatures and/or pressures. For instance, residence times in the first reactor and/or second reactor can vary between 1 second to 3 hours. In various implementations, a residence time in the first reactor and/or second can be about 1 second; about 5 seconds; about 10 seconds; about 15 seconds; about 20 seconds; about 30 seconds; about 45 seconds; about 60 seconds; about 75 seconds; about 90 seconds; about 105 seconds; about 120 seconds; about 3 minutes; about 5 minutes; about 10 minutes; about 20 minutes; about 30 minutes; about 45 minutes; about 60 minutes; about 90 minutes; about 120 minutes; about 150 minutes; or about 180 minutes. A residence time in the first reactor and/or second can be between 1 second and 15 seconds; between 15 seconds and 45 seconds; between 45 seconds and 2 minutes; between 1 minute and 25 minutes; between 25 minutes and 45 minutes; or between 1 minute and 45 minutes.

### IV. Example System Arrangements

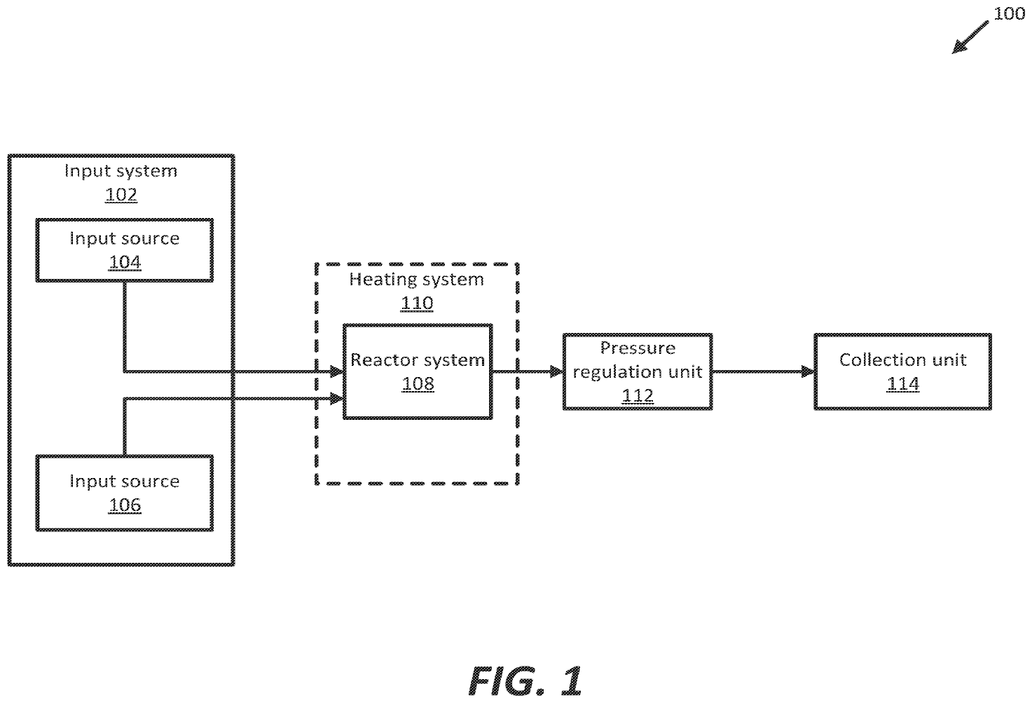

FIG. 1 is a schematic diagram of example aldehyde generation system 100. Broadly, example system 100 includes input system 102, reactor system 108, and heating system 110. A pressure regulation unit 112 can be part of reactor system 108 or positioned downstream from reactor system 108. Reactor system 108 can provide generated products to collection unit 114. Other embodiments can include more or fewer components.

Input system 102 provides various chemical constituents to reactor system 108. Input system 102 includes input source 104 and input source 106. Each of input source 104 and input source 106 includes one or more pump apparatus configured to provide various components at desired ratios to reactor system 108. Input source 104 and input source 106 can be configured to provide input streams to reactor system 108 such that a residence time of the reactor system is 1 minute to 3 hours. Other possible residence times are discussed above.

Typically, input source 104 provides an input stream including catalyst, ligand, and organic solvent. In some implementations, input source 104 also provides alkene substrate in liquid form. Example catalysts include rhodium, iron, and cobalt; an example ligand is BISBI; and example liquid alkene substrates include C₅-C₈ alkenes. Other examples are possible. In some instances, catalyst and ligand are pre-mixed, and then liquid alkene substrate can added to the catalyst-ligand mixture. In those instances, a first pump unit may provide the catalyst-ligand mixture and a second pump unit may provide the liquid alkene substrate.

Typically, input source 106 provides an input stream including carbon monoxide (CO) and hydrogen gas (H₂). In some implementations, input source 106 also provides a gaseous alkene substrate (e.g., C₃ and C₄ alkenes). In some instances, separate pump units are used to provide each of the carbon monoxide (CO), hydrogen gas (H₂), and gaseous alkene substrate. In some implementations, carbon monoxide (CO) and hydrogen gas (H₂) are pre-mixed with gaseous alkene substrate prior to entry into reactor system 108. In some instances, gaseous alkene substrate is not pre-mixed with carbon monoxide (CO) and/or hydrogen gas (H₂). One or more mass flow controller units may be used to adjust a flow rate of carbon monoxide (CO), hydrogen gas (H₂), and/or gaseous alkene substrate.

Reactor system 108 includes a first reactor and a second reactor, where the second reactor is positioned within the first reactor. Components provided by input system 102 undergo one or more chemical reactions in reactor system 108 to generate one or more aldehyde products. The second reactor is gas-permeable and the first reactor is gas-impermeable.

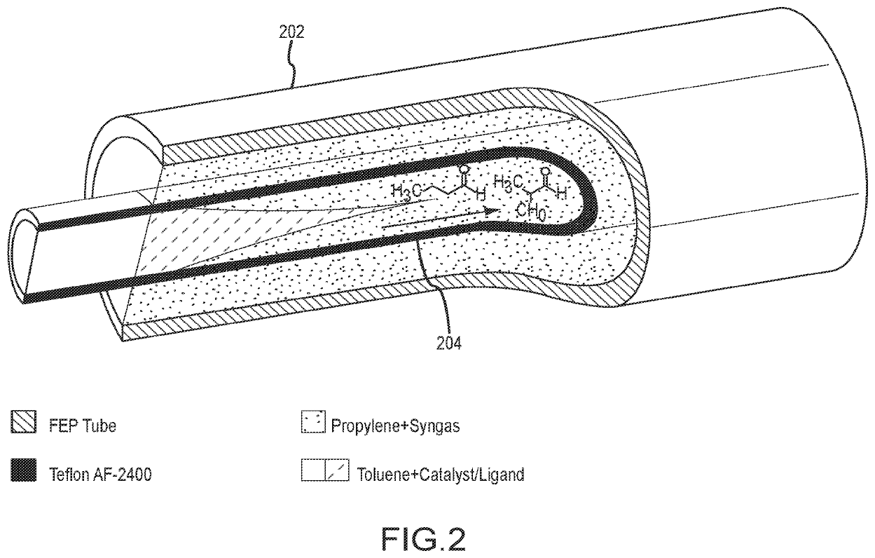

In some instances, the first reactor and the second reactor have a tubular shape. FIG. 2 shows a cut-away view showing a portion of example first reactor 202 and second reactor 204. Arrows indicate directions of flow, which is co-current in the embodiment shown. First reactor 202 is shown as being made of fluorinated ethylene propylene (FEP), and second reactor 204 is shown as being made of Teflon AF-2400. In the embodiment shown in FIG. 2, propylene and syngas is provided to first reactor 202 and toluene, catalyst, and ligand are provided to second reactor 204.

Referring again to FIG. 1, in some instances, input source 104 is in fluid communication with the first reactor and input source 106 is in fluid communication with the second reactor. In some instances, input source 104 is in fluid communication with the second reactor and input source 106 is in fluid communication with the first reactor. In some instances, flow in the first reactor is co-current with flow in the second reactor. In some instances, flow in the first reactor is counter-current to flow in the second reactor.

In some implementations, reactor system 108 includes a plurality of reactors operating in parallel. In some instances, a single input system 102 provides input source 104 and input source 106 to each reactor operating in parallel, such that each reactor receives the same ratio of chemical components.

Heating system 110 controls temperature within reactor system 108. Typically, heating system 110 controls temperature within reactor system 108 to be between 80° C. and 120° C., but other temperatures are contemplated. In some implementations, heating system 110 includes a first plate and a second plate, where the first plate and/or the second plate are heated.

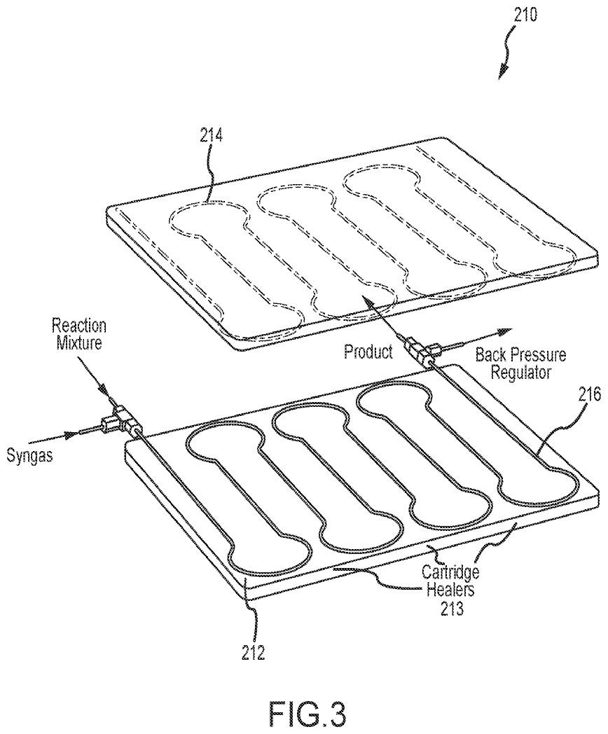

FIG. 3 shows a perspective, partially exploded view of example heating system 210. Heating system 210 includes first plate 212 that includes cartridge heaters 213 and second plate 214. A tube-in-tube reactor 216 is shown positioned between first plate 212 and second plate 214. In the embodiment shown, a length of tube-in-tube reactor 216 is 2 meters.

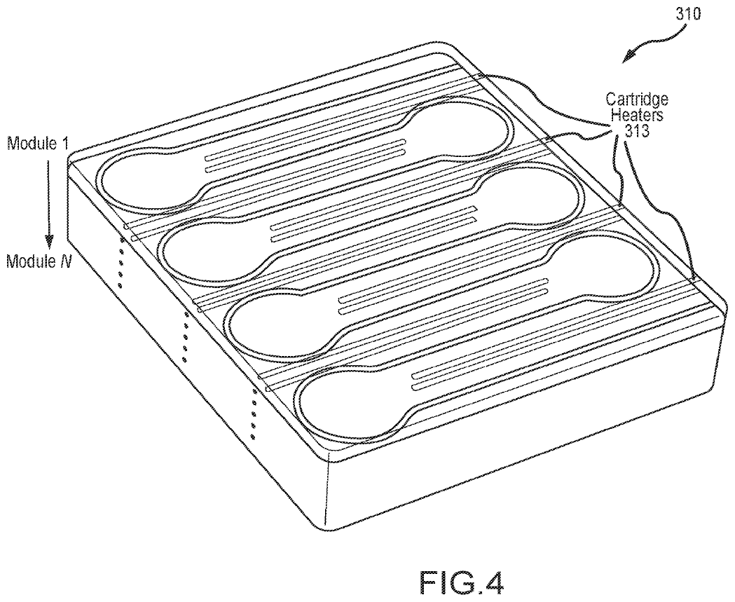

FIG. 4 shows a perspective view of example heating system 310. Heating system 310 is configured for holding a plurality of reactor modules, shown as 1 through N. A plurality of cartridge heater 313 locations are also shown.

Referring again to FIG. 1, pressure regulation unit 112 can be configured to control pressure within reactor system 108. An example pressure regulation unit 112 is a back pressure regulator, which may be manual or digital. Typically, pressure regulation unit 112 monitors and adjusts pressure within reactor system 108 to be less than 500 psig, less than 300 psig, less than 150 psig, or less than 100 psig. Other possible pressures are discussed in greater detail above.

Pressure regulation unit 112 may be positioned within heating system 110 or outside of heating system 110. In implementations where reactor system 108 includes a plurality of reactors, each reactor may include a pressure regulation unit 112.

One or more output streams from reactor system 108 can be provided to collection unit 114. In some instances, collection unit 114 is pressurized. For example, nitrogen (N₂) gas may be provided to collection unit 114 to maintain desired pressures.

In some instances, example system 100 can include one or more temperature monitoring apparatus, one or more pressure monitoring apparatus, and/or one or more mass flow apparatus. For instance, input system 102 may include one or more mass flow controllers, heating system 110 can include one or more temperature monitoring apparatus and corresponding control apparatus to adjust temperatures of heating system 110, and reactor system 108 can include one or more temperature monitoring apparatus. Other monitoring and flow regulation devices are possible. One or more controller units may be in electrical communication with one or more of the aforementioned monitoring and flow regulation devices and thereby adjust flow rates, mole ratios of chemical reagents, temperatures within reactor system 108, residence times within reactor system 108, and/or pressures within reactor system 108.

### V. Example Methods of Operation

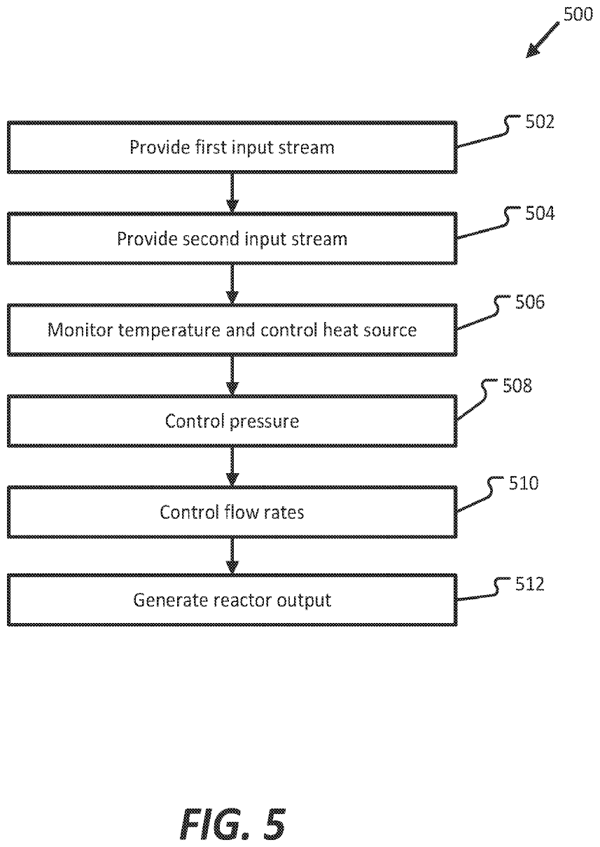

FIG. 5 shows example method 500 for generating aldehydes. As shown, example method 500 includes providing a first input stream 502, providing a second input stream 504, monitoring temperature and controlling a heat source (operation 506), controlling pressure (operation 508), controlling flow rates (operation 510), and generating reactor output (operation 512). Other embodiments can include more or fewer operations.

Method 500 begins by providing a first input stream (operation 502) and a second input stream (operation 504) to a reactor system. The first input stream includes catalyst, a ligand, and an organic solvent. In some implementations, the first input stream can include liquid alkene substrate. Example catalysts, ligands, liquid alkene substrates, and organic solvents are discussed in greater detail above. In some instances, the ligand and catalyst are pre-mixed before combining with the liquid alkene substrate and/or organic solvent.

The second input stream includes a mixture of carbon monoxide (CO) and hydrogen gas (H₂). In some instances, the second input stream can include a gaseous alkene substrate. The second input stream can have a molar ratio of CO to H₂ of 10:1-1:1-1:0. Other ratios are discussed in greater detail above.

As discussed in greater detail above, the reactor system includes a first reactor and a second reactor, where the second reactor is positioned within the first reactor. The first reactor is gas-impermeable and the second reactor is gas-permeable. Some implementations include a plurality of reactor systems operating in parallel, and, in those instances, the first input stream and the second input stream can be provided to each reactor in the reactor system.

In some instances, the first input stream is provided to the first reactor and the second input stream is provided to the second reactor. In some instances, the first input stream is provided to the second reactor, and the second input stream is provided to the first reactor.

During operation, temperature in the reactor system is monitored and adjusted as necessary (operation 506). A heating source, such as heating system 110 described with reference to FIG. 1, can control temperature within the reactor system to be within a predefined range. For instance, heating system 110 may control the temperature within the reactor system to be 80° C. to 120° C. Other possible temperatures are discussed in greater detail above.

Pressure can also be controlled (operation 508) as desired during operation. Typically, operating pressures in the reactor system are controlled (operation 510) to be less than 500 psig; less than 110 psig; less than 100 psig; or less than 75 psig. Other possible operating pressures are discussed above. Pressures within the reactor system can be controlled with, for example, a pressure regulation unit 112 as described with reference to FIG. 1.

Flow rates of the first input stream and/or the second input stream can also be controlled (operation 510) as desired during operation. Typically, flow rates are controlled such that residence time in the reactor system is 1 second to 3 hours. Other possible residence times are discussed above.

Reactor output is generated (operation 512) during operation. Typically, operation of the reactor system is continuous, so the reactor system can continuously generate output. As noted above, reactor output streams include one or more aldehydes. In some instances, the aldehydes have a linear to branch aldehyde ratio greater than 15. Other ratios are possible and discussed in greater detail above.

### VI. Experimental Examples

Experiments were conducted with a single-droplet flow reactor arrangement and a continuous flow reactor arrangement. Aspects of single-droplet flow reactor arrangements and experimental results are discussed in Zhu, Cheng, et al. “Flow chemistry-enabled studies of rhodium-catalyzed hydroformylation reactions.” Chem. Commun., 2018, 54, 8567-8570, the entirety of which is hereby incorporated by reference. Various aspects of the exemplary continuous flow reactor arrangements and experimental results are discussed below.

### A. Continuous Flow Reactor Experimental Setup

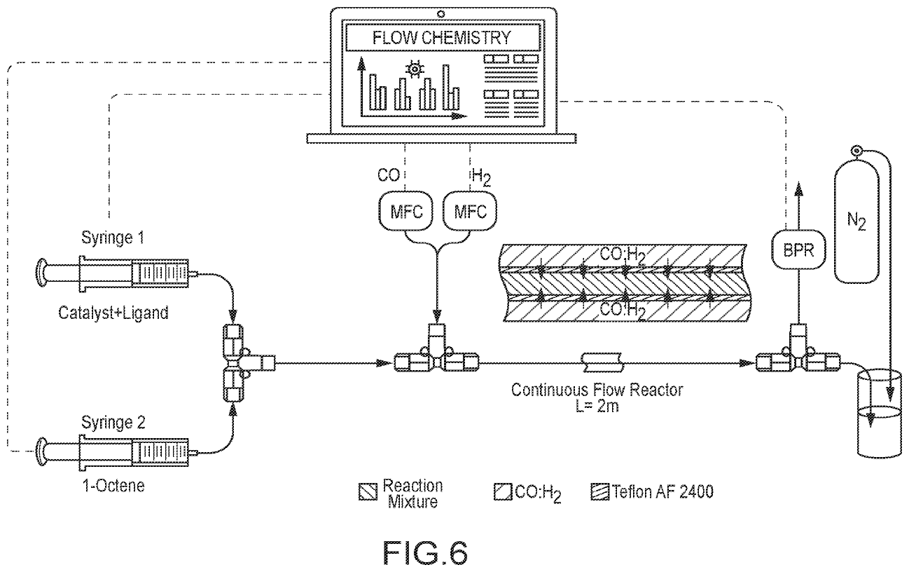

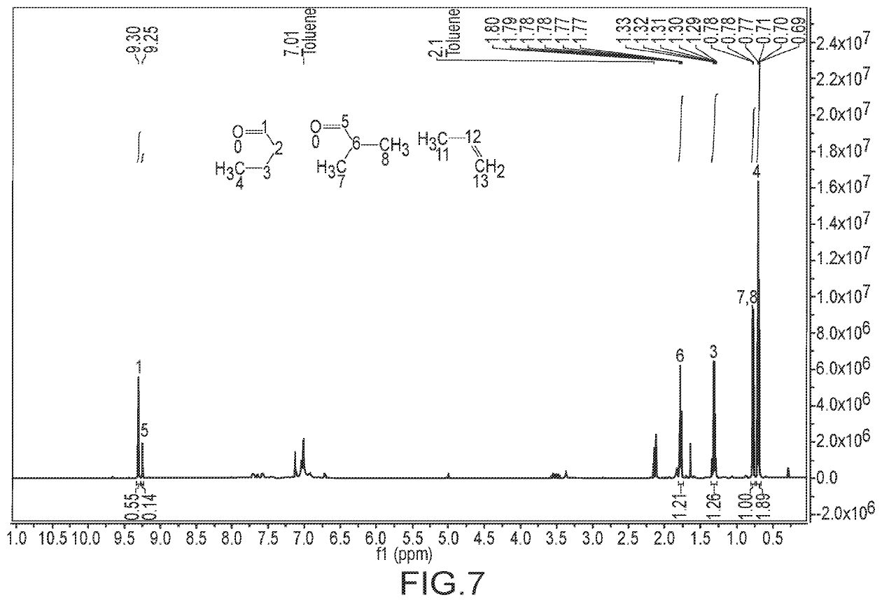

In order to demonstrate a direct utilization of the optimized hydroformylation results obtained using the single-droplet flow reactor (i.e., microliter-scale) in a larger scale aldehyde production, a continuous flow reactor module was designed and developed. A schematic illustration is shown in FIG. 6 and a portion of the system is shown in FIG. 7. Two reagent streams including (i) the catalyst and ligand mixture, and (ii) the substrate (1-octene) in toluene were fed to the continuous flow reactor using two syringe pumps (see syringe 1 and 2 in FIG. 6), and mixed at the first T-junction before flowing into the continuous flow tube-in-tube reactor.

The continuous flow hydroformylation reactions were efficiently carried out in the same tube-in-tube flow reactor configuration that was utilized for the single-droplet screening reactor. As shown in FIG. 6, the liquid stream containing the reaction mixture was continuously fed into the inner gas-permeable Teflon tubing (total volume: 1 mL), while the syngas mixture was continuously fed through the annular section between the inner (Teflon AF 2400) and outer (fluorinated ethylene propylene, FEP) tubing.

The CO and H₂ flow rates were controlled by two mass flow controllers (EL-FLOW, Bronkhorst), and the total syngas pressure in the continuous flow reactor was adjusted via a back-pressure regulator (EL-PRESS, Bronkhorst, FIG. 6). The reactor temperature was actively controlled using eight capillary heaters evenly distributed in the bottom and top CNC-machined aluminum plates (FIG. 6), operated with a PID temperature controller (Omega). The reaction time within the continuous flow reactor was controlled by adjusting the total liquid flow rate. The exiting liquid stream was collected into a pressurized vessel (FIG. 6) and analyzed off-line using gas chromatography (GC), HPLC, and nuclear magnetic resonance (NMR) spectroscopy. A Bruker 600 MHz instrument was used to analyze a sample of the product dissolved in 0.55 mL deuterated DMSO (dmso-d6). The continuous flow chemistry platform offered the possibility for scaling-out (i.e., numbering-up) of the hydroformylation reactor, while maintaining the same heat and mass transfer characteristics of the single-droplet flow reactor with a similar reactor geometry (e.g., inner and outer tubing diameter).

### B. Experiments and Discussion

1. Effect of Mass Transport Rate

A set of experiments studied the effect of mass transport rate in different flow reactor geometries (i.e., single-droplet and continuous flow reactors) on hydroformylation of 1-octene. Both hydroformylation reactions were conducted under similar reaction conditions using Rh(acac)(CO)₂ as the source of Rh and BISBI as the ligand for the catalytic system.

TABLE 1
Single-droplet vs continuous flow hydroformylation of 1-octene.^{a}

| | Synthesis | 1-Octene | | | | Mass |
|---|---|---|---|---|---|---|
| | Condition/ | Conver- | 2-Octene | Aldehyde | L/B | Bal- |
| Entry | Analysis | sion/% | Yield/% | Yield/% | Ratio | ance/% |
| 1 | Single | 87 | 11 | 74 | 17 | 98 |
| | droplet/LC | | | | | |
| 2 | Continuous | 93 | 19 | 72 | 19 | 98 |
| | flow/GC | | | | | |

^{a}General reaction conditions: 1-octene concentration: 0.5M, Rh(acac)(CO)₂ concentration: 0.5 mM, BISBI/Rh ratio: 2.30:1.0, syngas pressure (1:1):50.0 psig, syngas flow rate: 0.3 mL min⁻¹, droplet oscillation flowrate: 100 μL min⁻¹, temperature: 95° C., and reaction time: 20 min.

Similar chemo- and regioselectivity values of the hydroformylation reactions conducted using the single-droplet and continuous flow reactors (Entries 1-2, Table 1) indicate that the mass transport characteristics and catalytic performance of the BISBI/Rh system are similar between the single-droplet (15 μL) and the continuous flow reactors. We also investigated the effect of the outer tubing material on the hydroformylation of 1-octene by replacing the outer FEP tubing with a stainless steel tubing (supporting information S1.2). Similar aldehyde yield and regioselectivity obtained for both stainless steel and FEP outer tubing suggest that the FEP tubing does not affect the in-flow catalytic performance of the active Rh catalyst in hydroformylation of 1-octene.

### 2. Effect of Residence Time

Following the validation of the results of the single-droplet screening reactor with the continuous flow reactor, another study evaluated the effect of residence (reaction) time on the hydroformylation of 1-octene (1-30 min) at the relatively low total syngas pressure of 50 psig. As shown in Table 2, at 20 min residence time, an overall aldehyde yield of 72% with regioselectivity of 19 was observed (Entry 4, Table 2). This result is in agreement with the kinetic studies of phosphine-based ligands previously reported in flow.

TABLE 2
Continuous flow synthesis of aldehyde at different reaction times.^{a}

| | | 1-Octene | | | | Mass |
|---|---|---|---|---|---|---|
| | Residence | Conver- | 2-Octene | Aldehyde | L/B | Bal- |
| Entry | Time/min | sion/% | Yield/% | Yield/% | Ratio | ance/% |
| 1 | 1 | 10 | 3.4 | 5.1 | 5 | 98 |
| 2 | 5 | 44 | 9.5 | 32 | 10 | 97 |
| 3 | 10 | 63 | 15 | 44 | 18 | 96 |
| 4 | 20 | 93 | 19 | 72 | 19 | 98 |
| 5 | 30 | 97 | 27 | 67 | 21 | 97 |

^{a}General reaction conditions: 1-octene concentration: 0.5M, Rh(acac)(CO)₂ concentration: 0.5 mM, BISBI/Rh ratio: 2.50:1.0, syngas pressure (1:1):50.0 psig, syngas flow rate: 0.3 mL min⁻¹ and reaction time: 1-30 min.

The developed continuous hydroformylation process of olefins at 50.0 psig syngas pressure enables low operation cost, access to a reconfigurable synthesis platform with intensified mass transport rates which is more dimensionally accessible than conventional bulky autoclave reactors.

### 3. Catalyst Performance Assessment

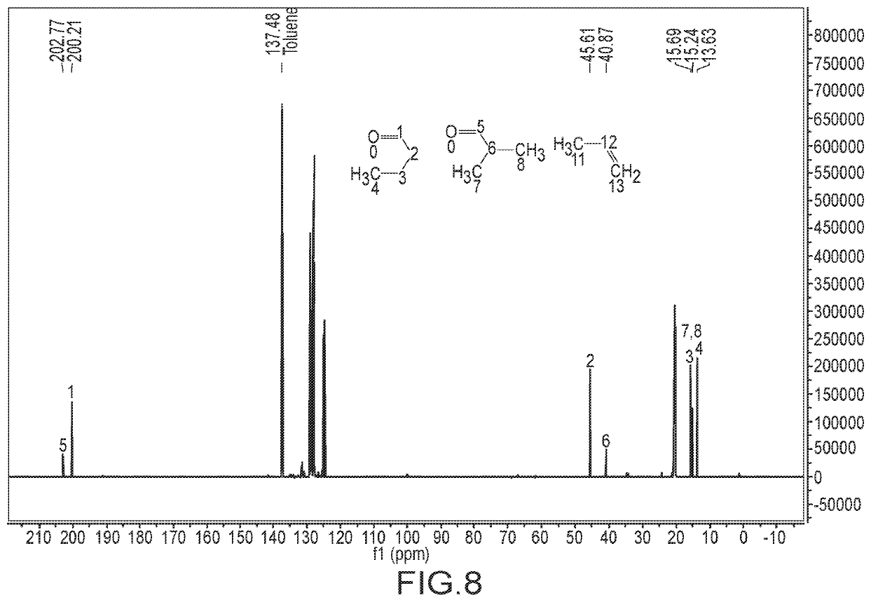

The developed continuous flow reactor was used to assess the catalytic performance of (i) fluorophosphite ligand in Rh-catalyzed hydroformylation reactions and (ii) BISBI/Rh system in hydroformylation of propylene. Example results are shown in Table 3 and FIG. 7 and FIG. 8 show nuclear magnetic resonance (NMR, proton and carbon respectively) spectroscopy for hydroformylation of propylene. The expected products, namely linear and branched butyraldehyde, are shown in the figures and the corresponding protons and carbons are marked on their respective NMR peaks. The two figures confirm that both the linear and branched butyraldehydes are present in the reaction product and the peak areas show a linear to branched selectivity of 3.8.

Relatively high L/B ratios (3.2-3.8) under high aldehyde yields (64-82%) were observed at various syngas compositions of H₂:CO with fluorophosphite/Rh system. Similar to 1-octene, decreasing the syngas pressure from 200 psig to 100 psig increased the L/B selectivity of the aldehyde product of propylene hydroformylation, conducted in the continuous tube-in-tube flow reactor, from 3.8 to 32 (see Table 3, below). However, further decrease in the syngas pressure from 100 psig to 50 psig, did not significantly affect the L/B ratio.

TABLE 3
Continuous flow hydroformylation of propylene.^{a}

| | Syngas | |
|---|---|---|
| S. No | Pressure/psi | L/B Ratio |
| 1 | 200 | 3.8 ± 1.9% |
| 2 | 100 | 32.8 ± 4.6% |
| 3 | 50 | 29.5 ± 6.0% |

^{a}General reaction conditions: Rh(CO)₂(acac) concentration: 0.5 mM, BISBI/Rh ratio: 2.30:1.0, syngas pressure (1:1): 50-200 psig, syngas flow rate: 0.3 mL min⁻¹, temperature: 95° C., and reaction time: 20 min.

Given the relatively high chemo- and regioselectivity of the hydroformylation reactions (Reaction I, above) conducted at low syngas pressures in the tube-in-tube flow reactor, we studied the catalytic resting state of the hydroformylation reaction using the H/D scrambling experiments. An accepted hydroformylation mechanism is a five-coordinated complex Rh(H)(CO)₂(PP), with the PP ligand coordinating in either a bis-equatorial (ee) or an equatorial-apical (ea) mode. It is theorized that ligand coordination mode strongly influences the regioselectivity during the hydroformylation catalytic cycle and is severely hampered by unclear coordination (mixture of ee and ea) of the ligand to the metal center.

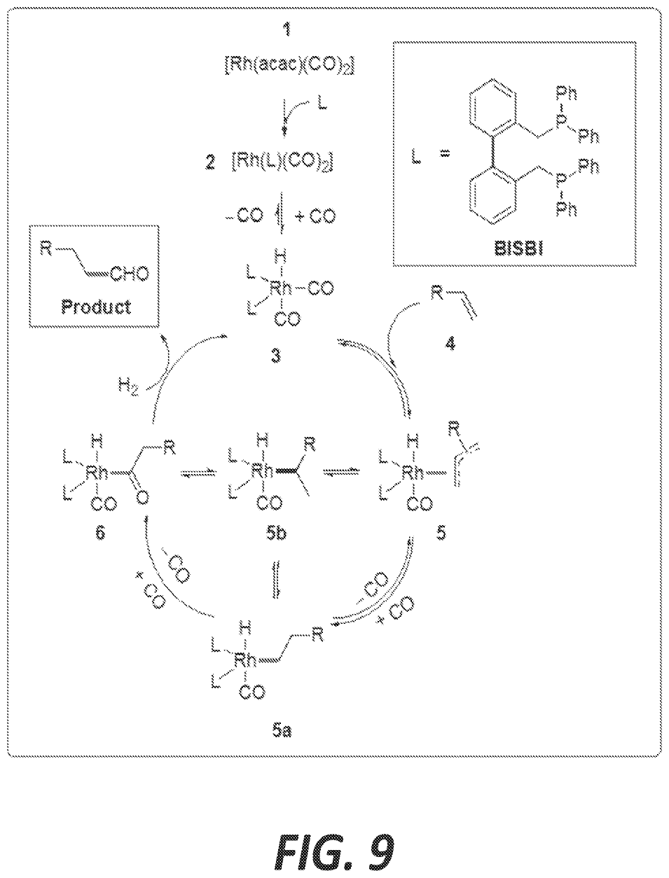

In order to explore the reaction mechanism of hydroformylation reactions in the continuous flow reactor, we studied the reversibility of migratory insertion of the Rh-alkyl intermediate (5, FIG. 9, Scheme II).

Scheme II, shown in FIG. 9, shows a plausible mechanism of Rh-catalyzed hydroformylation of alkenes in a tube-in-tube reactor using BISBI as the ligand. It is noted that an equilibrium exists between compounds 5a and 5b, respectively, which lead towards linear and branched aldehyde products; the equilibrium, however, is largely shifted toward compound 5a, thereby resulting in the linear aldehyde as major product.

If the regioselectivity of the produced aldehydes were mainly affected by the inability of linear intermediate species to deliver linear aldehydes, due to hydrogenolysis or the challenging insertion of CO, then one would expect highly reversible formation of the linear Rh-alkyl (5) compound. Thus, most of the formed branched aldehydes would come from the alkene (4) entering the catalytic hydroformylation cycle more than once.

To study the hydroformylation reaction mechanism in flow, reactions were conducted using deuterated starting material (styrene), while catalytic activation was performed similar to the previously optimized condition (Scheme II). Previously, a kinetic isotope effect (KIE) promoting elimination of H compared to D (2:1) has been demonstrated.

Considering the kinetic isotope effect in the elimination of β-hydride/deuteride, the experimentally-obtained 21% yield of 8a (Scheme II) underestimates the amount of branched aldehyde that is formed from a linear alkyl intermediate.

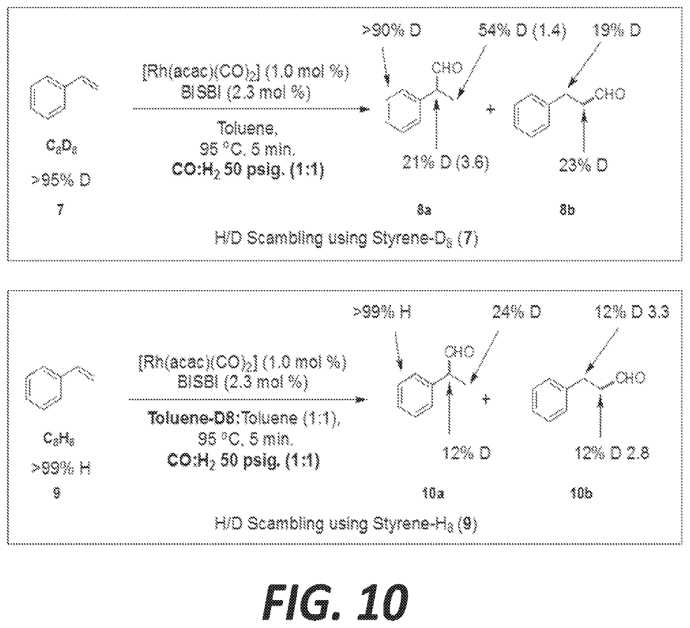

Scheme III, shown in FIG. 10, shows H/D scrambling study of Rh-catalyzed hydroformylation of alkenes in a tube-in-tube flow reactor using deuterated styrene (7) and styrene (9) as substrates, and BISBI as ligand.

Although the KIE of this hydroformylation reaction cannot be directly measured, KIE values exceeding 25 would be required to support a possible branched aldehyde formation route through the reversible linear Rh-alkyl specie (5a). Therefore, the results of the H/D scrambling experiments suggest that the regioselectivity of hydroformylation reactions conducted in the tube-in-tube flow reactor is mainly controlled at the early-stage of the catalytic cycle. This is in agreement with the previous mechanistic studies of the Rh-catalyzed hydroformylation reactions.

To summarize, it is theorized that these experiments demonstrate the ability to perform hydroformylation of 1-octene at a relatively low syngas pressure (50 psig) in a continuous flow reactor with intensified heat and mass transfer rates without any start-up/shut-down time delays that are typical of batch reactors. Furthermore, the reaction optimization results obtained using the single-droplet screening platform were directly transferred to the continuous flow synthesis reactor using a similar reactor configuration. A mechanistic study was conducted using H/D scrambling experiments in flow, and a plausible reaction mechanism was proposed. The facile operation of the hydroformylation reaction at low syngas pressures enabled by the intensified continuous flow reactor may reduce the total operation cost and capital expenditure (CAP-EX) required for large-scale aldehyde synthesis.

It is understood that the foregoing detailed description and accompanying examples are merely illustrative and are not to be taken as limitations upon the scope of the disclosure. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use, may be made without departing from the spirit and scope of the disclosure.

1. A method for generating aldehydes, the method comprising:
providing a first input stream to a reactor system, the first input stream including a catalyst, a ligand, and an organic solvent,
wherein the ligand is 2,2′-Bis(diphenylphosphinomethyl)-1,1′-bipheny (BISBI);
providing a second input stream to the reactor system, the second input stream including a mixture of carbon monoxide (CO) and hydrogen gas (H₂);
providing an alkene substrate to the reactor system, the alkene substrate being in either gaseous form or liquid form, the liquid form of the alkene substrate being provided with the first input stream, the gaseous form of the alkene substrate being provided with the second input stream,
wherein the alkene substrate is a C₃₋₈ alkene;
wherein the reactor system includes:
a first reactor and a second reactor, the second reactor being positioned within the first reactor;
the first reactor being gas-impermeable; and
the second reactor being gas-permeable;
monitoring a temperature within the first reactor;
controlling a heating source such that the temperature within the reactor system is 80° C. to 120° C.;
controlling a pressure within the reactor system to be less than 150 psig;
controlling a first input stream flow rate and a second input stream flow rate such that a residence time in the second reactor is 1 second to 3 hours; and
generating a reactor output stream including the aldehydes.

2. The method according to claim 1, wherein the first input stream is provided to the first reactor, and the second input stream is provided to the second reactor.

3. The method according to claim 1, wherein the first input stream is provided to the second reactor, and the second input stream is provided to the first reactor.

4. The method according to claim 1, wherein the catalyst is rhodium, iron, or cobalt.

5. The method according to claim 1, wherein the mixture of CO/H₂ has a molar ratio of CO to H₂ of 10:1-1:1-1:0.

6. The method according to claim 1, wherein the aldehydes have a linear to branch aldehyde ratio greater than 15.

7. The method according to claim 1, wherein the temperature is 90° C. to 110° C.;
wherein the residence time is 1 minute to 45 minutes; and
wherein the pressure is at least 50 psig and no more than 120 psig.

8. The method according to claim 1, wherein providing the first input stream and the second input stream is performed such that a flow in the first reactor is co-current with a flow in the second reactor.

9. The method according to claim 1, wherein providing the first input stream and the second input stream is performed such that a flow in the first reactor is counter-current with a flow in the second reactor.

10. The method according to claim 1, wherein the organic solvent is toluene.

11. The method according to claim 1, further comprising pre-mixing the ligand and the catalyst before mixing the ligand, catalyst, organic solvent, and the liquid form of the alkene substrate.

12. The method according claim 1, further comprising providing the first input stream and the second input stream to a plurality of reactor systems, where each reactor in the plurality of reactor systems operates in parallel.

13. The method according to claim 1, wherein the alkene substrate is 1-octene, hexene, pentene, or propylene.

14. A system for generating aldehydes, the system comprising:
a reactor system including a first reactor and a second reactor,
the second reactor being positioned within the first reactor;
the first reactor being gas-impermeable; and
the second reactor being gas-permeable;
a heating system configured to control a temperature within the reactor system to be 80° C. to 120° C.;
an input system configured to provide input streams to the reactor system such that a residence time of the reactor system is 1 minute to 1 hour,
wherein a first input stream includes a catalyst, a ligand, and an organic solvent;
wherein a second input stream includes a mixture of carbon monoxide (CO) and hydrogen gas (H₂);
wherein the input system provides an alkene substrate in either gaseous form or liquid form, the liquid form of the alkene substrate being provided with the first input stream, the gaseous form of the alkene substrate being provided with the second input stream;
a pressure regulation unit configured to control a reactor system pressure to be no greater than 110 psig;
a system outlet configured to discharge an output stream to a pressurized collection unit.

15. The system according to claim 14, wherein both the first reactor and the second reactor have a tubular shape.

16. The system according to claim 14, wherein the heating system includes a first plate and a second plate, at least one of the first plate and second plate being heated; and
wherein the reactor system is positioned between the first plate and the second plate.

17. The system according to claim 14, wherein the input system includes:
a first pump unit configured to provide the catalyst and the ligand;
a second pump unit configured to provide the liquid alkene substrate;
at least one mass flow controller unit configured to adjust a flow rate of the mixture of carbon monoxide (CO) and hydrogen gas (H₂).

18. The system according to claim 14, wherein the first input stream is provided to the first reactor, and the second input stream is provided to the second reactor.

19. The system according to claim 14, wherein the first input stream is provided to the second reactor, and the second input stream is provided to the first reactor.
